SEQUENCE LISTING (13 sequences)

SEQ ID NO: 1 - protein, 832 aa, synthetic construct
  REGION 1..832: note = Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 2 - protein, 833 aa, synthetic construct
  REGION 1..833: note = Description of Artificial Sequence: Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 3 - protein, 822 aa, synthetic construct
  REGION 1..822: note = Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 4 - protein, 381 aa, synthetic construct
  REGION 1..381: note = Description of Artificial Sequence: Synthetic polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGAEAAAKEAAAKEAAAKAGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 767 aa, synthetic construct
  REGION 1..767: note = Description of Artificial Sequence: Synthetic polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGAEAAAKEAAAKEAAAKAGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 59 aa, Drosophila sp.
QLRNDFQIHSVRGIKNPGDEDNGEPPANVKYVITGIILVIIALAFFGMVLSTQRKRAHG

SEQ ID NO: 7 - protein, 58 aa, Caenorhabditis sp.
GLPITEAMVAVPKRNEIDEGWSRSQVILFACIAFLAFGTVVAGVIAKNGPERSRKRKM

SEQ ID NO: 8 - protein, 53 aa, Sus sp.
SLNIPYKIEAVQSETVEPPPPPPLHFMYVAVVAFVLLFFVGCGVLLSRKRRRQ

SEQ ID NO: 9 - protein, 53 aa, Mus sp.
SLNIPYKIEAVKSEPVEPPLPSQLHLMYVAAAAFVLLFFVGCGVLLSRKRRRQ

SEQ ID NO: 10 - protein, 53 aa, Homo sapiens
SLNIPYKIEAVQSETVEPPPPAQLHFMYVAAAAFVLLFFVGCGVLLSRKRRRQ

SEQ ID NO: 11 - protein, 52 aa, Homo sapiens
QGTLSYPLVSVVSESLTPERTQLLYLLAVAVVIILFIILLGVIMAKRKRKHG

SEQ ID NO: 12 - protein, 52 aa, Homo sapiens
RLDFPYPLRDVRGEPLEPPEPSVPLLPLLVAGAVLLLVILVLGVMVARRKRE

SEQ ID NO: 13 - protein, 56 aa, Homo sapiens
EPLLPGPLLAVHPHAGTAPPANQLPWPVLCSPVAGVILLALGALLVLQLIRRRRRE

(19) United States
(12) Patent Application Publication
(10) Pub. No.: US 2024/0343777 A1
(43) Pub. Date: Oct. 17, 2024

(54) MODULAR SYNTHETIC RECEPTORS AND METHODS OF USE

(71) Applicant: Lung Biotechnology PBC, Silver Spring, MD (US)

(72) Inventors: Jintang Du, San Diego, CA (US); Nanna Yum, San Diego, CA (US); Michael Brown, San Diego, CA (US); Colin Exline, San Diego, CA (US); Sean Stevens, Del Mar, CA (US)

(73) Assignee: Lung Biotechnology PBC, Silver Spring, MD (US)

(21) Appl. No.: 18/756,911

(22) Filed: Jun. 27, 2024

Related U.S. Application Data
(62) Division of application No. 17/222,385, filed on Apr. 5, 2021, now Pat. No. 12,049,488.
(60) Provisional application No. 63/005,739, filed on Apr. 6, 2020.

(51) Int. Cl. C07K 14/705; C07K 14/55; C07K 16/30

(52) U.S. Cl. CPC C07K 14/70521; C07K 14/55; C07K 16/3084; C07K 2317/622; C07K 2319/03; C07K 2319/30

(57) ABSTRACT

Modular synthetic receptors are provided. The synthetic receptors may include an extracellular domain capable of binding to one or more ligand molecules and may be released from the synthetic receptor after binding, a transmembrane domain derived from the Notch receptor, and an intracellular domain which may have one or more functional activities when released from the synthetic receptor. A method of use for the synthetic receptors is also provided, wherein upon binding of the extracellular domain to a specific ligand, the synthetic receptor undergoes proteolytic cleavage to release either or both the extracellular and intracellular domains. The extracellular binding domain, if released, may continue to bind to its cognate ligand and may carry one or more additional functional activities and the intracellular domain, if released, may stimulate or inhibit one or more intracellular activities.

### CROSS-REFERENCE TO RELATED APPLICATIONS

This application is a Divisional of U.S. application Ser. No. 17/222,385, filed Apr. 5, 2021, which claims priority to U.S. Provisional Application No. 63/005,739, filed Apr. 6, 2020, the entire contents of which are incorporated herein.

### SEQUENCE LISTING

The instant application contains a Sequence Listing which has been submitted electronically in XML format and is hereby incorporated by reference in its entirety. Said XML copy, created on Jun. 25, 2024, is named 080618-2438_SL.xml, and is 18,063 bytes in size.

### TECHNICAL FIELD

The present application relates to novel receptors, specifically Notch or CTLA-4 synthetic receptors designed for transplantation, oncology, and autoimmune therapies.

### BACKGROUND

Mammalian cells have transmembrane receptors that enable recognition of extracellular molecules and induce intracellular responses. The Notch receptor is an evolutionarily-conserved family of signaling receptors that utilize proteolytic cleavage to release extra- and intracellular domains in response to engagement with cognate ligands. The ability to undergo proteolytic cleavage is contained within a limited region of the Notch receptor, which contains the transmembrane domain and recognition sites for the cleavage event. The ability to undergo cleavage in response to ligand engagement is transferable, allowing the creation of synthetic receptors with a variety of ligand specificities which, upon binding, can release a variety of engineered extracellular and intracellular subdomains.

### SUMMARY

The present disclosure provides a synthetic receptor comprising at least three domains. In certain aspects of the disclosure, the synthetic receptor may include: a) at least one domain comprising an extracellular domain configured to specifically bind to one or more ligands and to optionally release from the synthetic receptor after binding with said ligand, b) at least one domain comprising a transmembrane domain comprising or derived from a Notch receptor, and c) at least one domain comprising an intracellular domain configured to optionally initiate one or more functional activities when released from the synthetic receptor.

In some embodiments, upon binding an extracellular domain to a specific ligand, the synthetic receptor may undergo proteolytic cleavage to release either or both the extracellular domain and the intracellular domain. The extracellular binding domain may continue to bind to a cognate ligand and carry out one or more functional activities even if released. Functional activities can include at least one of antibody-dependent cellular cytotoxicity, complement-dependent cytotoxicity, apoptosis, or an enzymatic function. Further, by way of illustration and not limitation, the activity can be at least one of blockade or induction of protein-protein interactions or secretion of extracellular functional molecules. In some embodiments, extracellular functional molecules may be at least one of cytokines or chemokines.

The intracellular domain of the synthetic receptor may stimulate or inhibit one or more intracellular activities if released. In certain aspects of the disclosure, the intracellular domain is a secreted protein which stimulates or inhibits one or more extracellular activities if released.

These extracellular activities can include at least one of, but are not limited to, signaling, trafficking, adhesion, blockade of protein-protein interactions and/or stability. The one or more intracellular activities comprise at least one of signaling, gene expression, trafficking, and/or stability.

The extracellular domain may comprise an antibody or a fragment thereof. For example, in some embodiments, the extracellular domain may be a single chain variable fragment (scFV) molecule of an antibody that binds glycolipid disialoganglioside (GD2) fused to an Fc region of human IgG1.

The intracellular activity can also include at least one secreted fusion protein of a human CTLA4 extracellular domain fused to a wild type or modified Fc region of a human immunoglobulin G (IgG). In certain embodiments and by way of illustration, the human IgG may be at least one of IgG1, IgG2, or IgG4

The intracellular domain activity may also include at least one transgene of a human interleukin.

For example, in certain embodiments, the intracellular activity includes at least one transgene encoding human interleukin 2. In yet another embodiment, the at least one transgene may encode human interleukin 12. In some embodiments, the at least one transgene may encode any one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40.

In certain aspects of the disclosure, Notch receptor of the synthetic receptor can be a member of a human Notch receptor family. In other embodiments of the synthetic receptors, the Notch receptor is a member of the Notch receptor family from at least one of fly, worm, pig, or mouse.

The receptor may comprise a human CD3-specific single chain Fv molecule fused to an Fc region of a human IgG1. In these embodiments, the receptor may include a polypeptide sequence having at least 85%, at least 90%, at least 95%, or at least 98% amino acid sequence identity to SEQ ID NO: 1 or SEQ ID NO: 3. The receptor also may include a mouse CD3-specific single chain Fv molecule fused to an Fc region of a human IgG1. In these embodiments, the transgene comprises a polypeptide sequence having at least 85% amino acid sequence identity to SEQ ID NO: 3.

In some embodiments, the synthetic receptor also may comprise a single chain Fv molecule derived from dinutuximab fused to the Fc region of human IgG1. In these embodiments, the receptor comprises a polypeptide sequence having at least 85% amino acid sequence identity to SEQ ID NO: 2.

In some embodiments, the receptor further comprises a transgene encoding a fusion protein made of the human CTLA4 extracellular domain fused to the Fc region of human IgG1.

In certain other embodiments, the receptor further comprises a transgene encoding a human interleukin 2 molecule fused to the Fc region of human IgG1. In these embodiments, the transgene comprises a polypeptide sequence having at least 85% amino acid sequence identity to SEQ ID NO: 4.

In further embodiments, the receptor comprises a transgene encoding an engineered single chain human interleukin 12 molecule fused to the Fc region of human IgG1. In these embodiments, the transgene comprises a polypeptide sequence having at least 85% amino acid sequence identity to SEQ ID NO: 5.

A method of modulating an activity of a target cell using a synthetic receptor is also provided herein. The method may include contacting the synthetic receptor with a receptor on the target cell, allowing cleavage of the synthetic receptor while the extracellular binding domain remains bound to the target cell and releasing the intracellular domain into the nucleus where it induces expression of a gene. In some aspects of the disclosure, the extracellular domain may be an antibody or a fragment thereof. In certain embodiments, the extracellular domain also may be a single chain Fv molecule of an antibody that binds glycolipid disialoganglioside fused to an Fc region of human IgG1.

The intracellular activity of the method can include at least one fusion protein of a human CTLA4 extracellular domain fused to a wild type or modified Fc region of human IgG. In some embodiments, the IgG may comprises IgG1, IgG2, or IgG4. The intracellular activity also may include at least one transgene encoding a human interleukin. In some embodiments, the human interleukin is human IL2. In certain other embodiments, the intracellular activity includes at least one transgene encoding human IL12. In some embodiments, the at least one transgene may encode any one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40.

Contacting the synthetic receptor with a receptor on the target cell may comprise administering the synthetic receptor to a subject suffering from cancer. Contacting further may include administering the synthetic receptor to a subject suffering from an autoimmune disorder. Contacting further may include administering the synthetic receptor to a subject after an allotransplant or xenotransplant.

### BRIEF DESCRIPTION OF THE DRAWINGS

FIG. 1 illustrates a schematic diagram of a synthetic receptor of the present disclosure.

FIG. 2A-2E show results of polyacrylamide gel electrophoresis demonstrating the production of correctly-sized products from synthetic constructs. FIG. 2A shows a synthetic receptor made of a human CD3-specific single chain Fv molecule fused to the Fc region of human IgG1. FIG. 2B illustrates a synthetic receptor made of a single chain Fv molecule derived from dinutuximab (sold under the trademark Unituxin® (dinutuximab) by United Therapeutics Corp.) fused to the Fc region of human IgG1. FIG. 2C shows a transgene encoding Belatacept, a fusion protein made of the human CTLA4 extracellular domain fused to the Fc region of human IgG1. FIG. 2D depicts a transgene encoding human interleukin 2 molecule fused to the Fc region of human IgG1. FIG. 2E shows a transgene encoding an engineered single chain human interleukin 12 molecule fused to the Fc region of human IgG1.

FIG. 3 graphically illustrates flow cytometric analysis demonstrating that the transgene-encoded Belatacept made of the human CTLA4 extracellular domain fused to the Fc region of human IgG1 binds to porcine CD80/CD86 molecules.

FIG. 4 graphically shows function of a synthetic receptor with extracellular domain made of a human CD3-specific single chain Fv molecule fused to the Fc region of human IgG1 which recognizes CD3 and responds with expression and secretion of Belatacept. Porcine aortic endothelial cells engineered with the anti-hCD3 synthetic receptor and a responsive transgene encoding Belatacept were exposed to human Jurkat T cells, which express CD3 natively, for 48 hours and analyzed for expression of Belatacept. Results show that only cells expressing the synthetic receptor induce expression of Belatacept in the presence of human T cells, which would have the dual benefit of blockade of CD3 and CD80/86.

FIG. 5A-5B. FIG. 5A graphically shows IL-2Fc production when pig aortic endothelial cells expressing the dinutuximab (sold under the trade mark Unituxin®) synthetic receptor are co-cultured with CHP-134 cells, which express the target glycolipid disialoganglioside (GD2) natively, for 48 hours. FIG. 5B graphically shows scIL-12Fc production level when pig aortic endothelial cells expressing the Unituxin synthetic receptor are co-cultured with CHP-134 cells, which express the target glycolipid disialoganglioside (GD2) natively, for 48 hours. IL-2Fc and scIL12-Fc protein levels were quantified by ELISA.

FIG. 6A-6B. FIG. 6A graphically illustrates that the engineered human IL2-Fc protein encoded by the transgene functions at levels similar to native human IL2 in a CTLL-2 proliferation assay (relative fluorescence units). FIG. 6B shows results of a CTLL-2 proliferation assay wherein supernatants from porcine aortic endothelial cells engineered with the anti-GD2 synthetic receptor and a responsive transgene encoding human IL2-Fc cultured with or without human CHP-134 cells, which express the target glycolipid disialoganglioside (GD2) natively, for 48 hours. “Negative” refers to CTLL-2 cells alone and “Positive” refers to CTLL-2 cells cultured with purified human IL2-Fc protein.

FIG. 7 shows alignments of the transmembrane domain and flanking 1^{st} and 2^{nd} cleavage recognition sequences from Notch receptors of fly (dNotch) (SEQ ID NO: 6), worm (GLP-1) (SEQ ID NO: 7), pig (pNotch1) (SEQ ID NO: 8), mouse (mNotch1) (SEQ ID NO: 9) and human (hNotch1-4) (SEQ ID NOS 10-13, respectively, in order of appearance).

### DETAILED DESCRIPTION

### Definitions

As used herein and in the appended claims, singular articles such as “a,” “an,” “the,” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member.

The term “exemplary” as used herein refers to “serving as an example, instance, or illustration,” and not “preferred” or “advantageous over other embodiments”.

As used herein, “antibody-dependent cellular cytotoxicity” (ADCC), also referred to as antibody-dependent cell-mediated cytotoxicity, can refer to a mechanism of cell-mediated immune defense whereby an effector cell of an immune system actively lyses a target cell, whose membrane-surface antigens have been bound by specific antibodies. It is one of the mechanisms through which antibodies, as part of the humoral immune response, can act to limit and contain infection.

As used herein, “Belatacept” can refer to a soluble fusion protein, which links the extracellular domain of human cytotoxic T-lymphocyte-associated antigen 4 (CTLA4) to the modified Fc (hinge, CH2, and CH3 domains) portion of human immunoglobulin G1 (IgG1). Structurally, abatacept is a glycosylated fusion protein with a MALDI-MS molecular weight of 92,300 Da and it is a homodimer of two homologous polypeptide chains of 357 amino acids each. It is produced through recombinant DNA technology in mammalian CHO cells. The drug has activity as a selective co-stimulation modulator with inhibitory activity on T lymphocytes.

As used herein, “complement-dependent cytotoxicity” (CDC) can refer to an effector function of immunoglobulin, typically IgG and IgM antibodies. When they are bound to surface antigen on target cell (e.g., bacterial or viral infected cell), the classical complement pathway is triggered by bonding protein C1q to these antibodies, resulting in formation of a membrane attack complex (MAC) and target cell lysis.

As used herein, “dinutuximab” (sold under the trademark Unituxin® (dinutuximab) by United Therapeutics Corp.) is a GD2-binding monoclonal antibody indicated, in combination with granulocyte macrophage colony-stimulating factor (GM-CSF), interleukin-2 (IL-2), and 13-cis-retinoic acid (RA), for the treatment of pediatric patients with high-risk neuroblastoma who achieve at least a partial response to prior first-line multiagent, multimodality therapy.

As used herein, unless specified otherwise, “human interleukin” can refer to any one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40. Interleukin play essential roles in the activation and differentiation of immune cells, as well as proliferation, maturation, migration, and adhesion. They also have pro-inflammatory and anti-inflammatory properties.

### Synthetic Receptors

The synthetic receptors comprise a) at least one domain comprising an extracellular domain configured to specifically bind to one or more ligands and to optionally release from the synthetic receptor after binding with said ligand, b) at least one domain comprising a transmembrane domain derived from a Notch receptor, and c) at least one domain comprising an intracellular domain configured to optionally initiate one or more functional activities when released from the synthetic receptor.

In some embodiments, upon binding an extracellular domain to a specific ligand, the synthetic receptor may undergo proteolytic cleavage to release either or both the extracellular domain and the intracellular domain. The extracellular binding domain may continue to bind to a cognate ligand and carry out one or more functional activities even if released. Functional activities can include at least one of antibody-dependent cellular cytotoxicity, complement-dependent cytotoxicity, or an enzymatic function. The activity can be at least one of blockade or induction of protein-protein interactions or secretion of extracellular functional molecules. The antibody-dependent cellular cytotoxicity (ADCC), also referred to as antibody-dependent cell-mediated cytotoxicity, is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. In some embodiments, extracellular functional molecules may be at least one of cytokines or chemokines.

The intracellular domain of the synthetic receptor may stimulate or inhibit one or more intracellular activities or extracellular activities, if released.

In certain aspects of the disclosure, the intracellular domain can be a secreted protein which stimulates or inhibits extracellular activity. These extracellular activities can include at least one of, but are not limited to, signaling, trafficking, adhesion, blockade of protein-protein interactions and/or stability. The one or more intracellular activities comprise at least one of signaling, gene expression, trafficking, and/or stability.

The extracellular domain may comprise an antibody or a fragment thereof. For example, in some embodiments, the extracellular domain may be a single chain Fv molecule of an antibody that binds glycolipid disialoganglioside (GD2) fused to an Fc region of human IgG1.

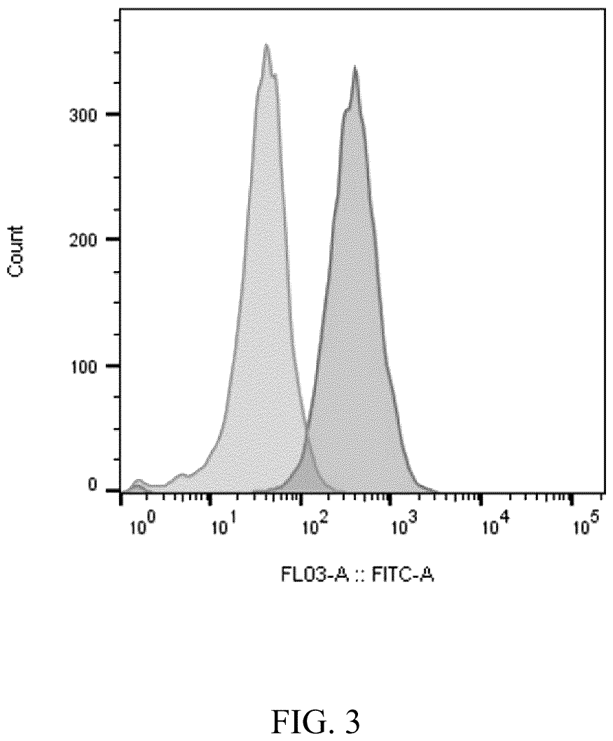

The intracellular activity can also include at least one secreted fusion protein of a human CTLA4 extracellular domain fused to a wild type or modified Fc region of a human immunoglobulin G (IgG). In certain embodiments and by way of illustration, the human IgG may be at least one of IgG1, IgG2, or IgG4. Referring to FIG. 3, a graphical depiction illustrates a flow cytometric analysis demonstrating that the transgene-encoded Betalacept made of human CTLA4 extracellular domain fused to the Fc region of human IgG1 binds to porcine CD80/CD86 molecules.

The intracellular domain activity may also include at least one transgene of a human interleukin.

For example, in certain embodiments, the intracellular activity includes at least one transgene encoding human interleukin 2. In yet another embodiment, the at least one transgene may encode human interleukin 12. In some embodiments, the at least one transgene may encode any one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40.

In certain aspects of the disclosure, Notch receptor subdomain of the synthetic receptor can be a member of the human Notch receptor family. In other embodiments of the synthetic receptors, the Notch receptor is a member of the Notch receptor family from at least one of fly, worm, pig, mouse, or human.

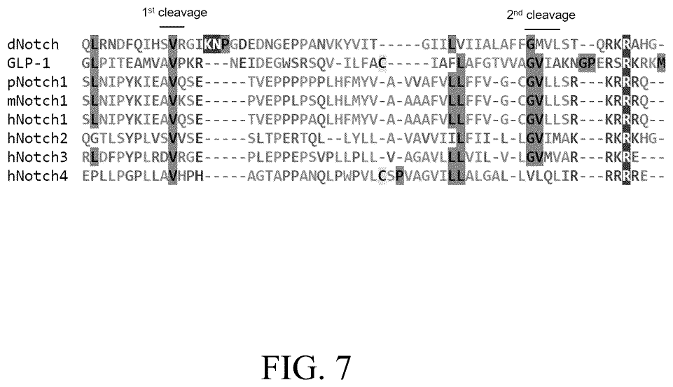

The receptor may comprise a human CD3-specific single chain Fv molecule fused to an Fc region of a human IgG1. In some embodiments, the receptor may include a polypeptide sequence having at least 85%, at least 90%, at least 95%, or at least 98% amino acid sequence identity to SEQ ID NO: 1, as disclosed in Table 1. The receptor may also comprise a mouse CD3-specific single chain Fv molecule fused to an Fc region of a human IgG1. In some embodiments, the receptor may include a polypeptide sequence having at least 85%, at least 90%, at least 95%, or at least 98% amino acid sequence identity to SEQ ID NO: 3, as disclosed in Table 3. Notch receptors may be derived from additional members of the human Notch receptor family (as shown in FIG. 7), or members of the Notch receptor family from other species which differ in sequence but retain the proteolytic cleavage functionality. FIG. 7 shows partial alignments of the first and second cleavage regions upon binding an extracellular domain to a specific ligand. Sequences from Notch receptors of fly (dNotch), worm (GLP-1), pig (pNotch1), mouse (mNotch1) and human (hNotch1-4) are shown.

In some embodiments, the synthetic receptor also may comprise a single chain Fv molecule derived from dinutuximab fused to the Fc region of human IgG1. In these embodiments, the receptor comprises a polypeptide sequence having at least 85%, at least 90%, at least 95%, or at least 98% amino acid sequence identity to SEQ ID NO: 2, as disclosed in Table 2.

In some embodiments, the receptor further comprises a transgene encoding a fusion protein made of the human CTLA4 extracellular domain fused to the Fc region of human IgG1.

In certain other embodiments, the receptor further comprises a transgene encoding a human interleukin 2 molecule fused to the Fc region of human IgG1. In these embodiments, the transgene comprises a polypeptide sequence having at least 85%, at least 90%, at least 95%, or at least 98% amino acid sequence identity to SEQ ID NO: 4, as disclosed in Table 4.

In further embodiments, the receptor comprises a transgene encoding an engineered single chain human interleukin 12 molecule fused to the Fc region of human IgG1. In these embodiments, the transgene comprises a polypeptide sequence having at least 85%, at least 90%, at least 95%, or at least 98% amino acid sequence identity to SEQ ID NO: 5. as disclosed in Table 5.

TABLE 1
Anti-CD3 hFc (SEQ ID NO: 1)

QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSQIVLTQSPAIMSA
SPGEKVTMTCSASSSVSYMNWYQQKSGTSPKRWIYDTSKLASGVPAHFR
GSGSGTSYSLTISGMEAEDAATYYCQQWSSNPFTFGSGTKLEINRILDY
SFGGGAGRDIPPPLIEEACELPECQEDAGNKVCSLQCNNHACGWDGGDC
SLNFNDPWKNCTQSLQCWKYFSDGHCDSQCNSAGCLFDGFDCQRAEGQC
NPLYDQYCKDHFSDGHCDQGCNSAECEWDGLDCAEHVPERLAAGTLVVV
VLMPPEQLRNSSFHFLRELSRVLHTNVVFKRDAHGQQMIFPYYGREEEL
RKHPIKRAAEGWAAPDALLGQVKASLLPGGSEGGRRRRELDPMDVRGSI
VYLEIDNRQCVQASSQCFQSATDVAAFLGALASLGSLNIPYKIEAVQSE
TVEPPPPAQLHFMYVAAAAFVLLFFVGCGVLLSRKRRRQLCIQKLMSRL
DKSKVINSALELLNEVGIEGLTTRKLAQKLGVEQPTLYWHVKNKRALLD
ALAIEMLDRHHTHFCPLEGESWQDFLRNNAKSFRCALLSHRDGAKVHLG
TRPTEKQYETLENQLAFLCQQGFSLENALYALSAVGHFTLGCVLEDQEH
QVAKEERETPTTDSMPPLLRQAIELFDHQGAEPAFLFGLELIICGLEKQ
LKCESGGPADALDDFDLDMLPADALDDFDLDMLPADALDDFDLDMLPG

TABLE 2
dinutuximab hFc (SEQ ID NO: 2)

EVQLLQSGPELEKPGASVMISCKASGSSFTGYNMNWVRQNIGKSLEWIG
AIDPYYGGTSYNQKFKGRATLTVDKSSSTAYMHLKSLTSEDSAVYYCVS
GMEYWGQGTSVTVSSGGGGSGGGGSGGGGSDVVMTQTPLSLPVSLGDQA
SISCRSSQSLVHRNGNTYLHWYLQKPGQSPKLLIHKVSNRFSGVPDRFS
GSGSGTDFTLKISRVEAEDLGVYFCSQSTHVPPLTFGAGTKLELKRILD
YSFGGGAGRDIPPPLIEEACELPECQEDAGNKVCSLQCNNHACGWDGGD
CSLNFNDPWKNCTQSLQCWKYFSDGHCDSQCNSAGCLFDGFDCQRAEGQ
CNPLYDQYCKDHFSDGHCDQGCNSAECEWDGLDCAEHVPERLAAGTLVV
VVLMPPEQLRNSSFHFLRELSRVLHTNVVFKRDAHGQQMIFPYYGREEE
LRKHPIKRAAEGWAAPDALLGQVKASLLPGGSEGGRRRRELDPMDVRGS
IVYLEIDNRQCVQASSQCFQSATDVAAFLGALASLGSLNIPYKIEAVQS
ETVEPPPPAQLHFMYVAAAAFVLLFFVGCGVLLSRKRRRQLCIQKLMSR
LDKSKVINSALELLNEVGIEGLTTRKLAQKLGVEQPTLYWHVKNKRALL
DALAIEMLDRHHTHFCPLEGESWQDFLRNNAKSFRCALLSHRDGAKVHL
GTRPTEKQYETLENQLAFLCQQGFSLENALYALSAVGHFTLGCVLEDQE
HQVAKEERETPTTDSMPPLLRQAIELFDHQGAEPAFLFGLELIICGLEK
QLKCESGGPADALDDFDLDMLPADALDDFDLDMLPADALDDFDLDMLPG

TABLE 3
anti-CD3 mFc (SEQ ID NO: 3)

QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSQIVLTQSPAIMSA
SPGEKVTMTCSASSSVSYMNWYQQKSGTSPKRWIYDTSKLASGVPAHFR
GSGSGTSYSLTISGMEAEDAATYYCQQWSSNPFTFGSGTKLEINRILDY
SFTGGAGRDIPPPQIEEACELPECQVDAGNKVCNLQCNNHACGWDGGDC
SLNFNDPWKNCTQSLQCWKYFSDGHCDSQCNSAGCLFDGFDCQLTEGQC
NPLYDQYCKDHFSDGHCDQGCNSAECEWDGLDCAEHVPERLAAGTLVLV
VLLPPDQLRNNSFHFLRELSHVLHTNVVFKRDAQGQQMIFPYYGHEEEL
RKHPIKRSTVGWATSSLLPGTSGGRQRRELDPMDIRGSIVYLEIDNRQC
VQSSSQCFQSATDVAAFLGALASLGSLNIPYKIEAVKSEPVEPPLPSQL
HLMYVAAAAFVLLFFVGCGVLLSRKRRRQLCIQKLMSRLDKSKVINSAL
ELLNEVGIEGLTTRKLAQKLGVEQPTLYWHVKNKRALLDALAIEMLDRH
HTHFCPLEGESWQDFLRNNAKSFRCALLSHRDGAKVHLGTRPTEKQYET
LENQLAFLCQQGFSLENALYALSAVGHFTLGCVLEDQEHQVAKEERETP
TTDSMPPLLRQAIELFDHQGAEPAFLFGLELIICGLEKQLKCESGGPAD
ALDDFDLDMLPADALDDFDLDMLPADALDDFDLDMLPG

TABLE 4
IL2-Fc (SEQ ID NO: 4)

APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFCQSIISTLTGGAEAAAKEAAAKE
AAAKAGGDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSK
LTVDKSRWQQGNVFSCSVMHEALHNHYTQKSLSLSPGK

TABLE 5
scIL12Fc (SEQ ID NO: 5)

IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSGGGSGGGSGGGSGGGSRNLPVATPDPGMFPCLHEISQ
NLLRAVSNMLQKARQTLEFYPCTSEEIDHEDITKDKTSTVEACLPLELT
KNESCLNSRETSFITNGSCLASRKTSFMMALCLSSIYEDLKMYQVEFKT
MNAKLLMDPKRQIFLDQNMLAVIDELMQALNFNSETVPQKSSLEEPDFY
KTKIKLCILLHAFRIRAVTIDRVMSYLNASGGAEAAAKEAAAKEAAAKA
GGDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDK
SRWQQGNVFScSVMHEALHNHYTQKSLSLSPGK

### Methods of Use and Treatment

A method of modulating an activity of a target cell using a synthetic receptor is also provided herein. The method may include contacting the synthetic receptor with a receptor on the target cell, allowing cleavage of the synthetic receptor while the extracellular binding domain remains bound to the target cell and releasing the intracellular domain into the nucleus where it induces expression of a gene.

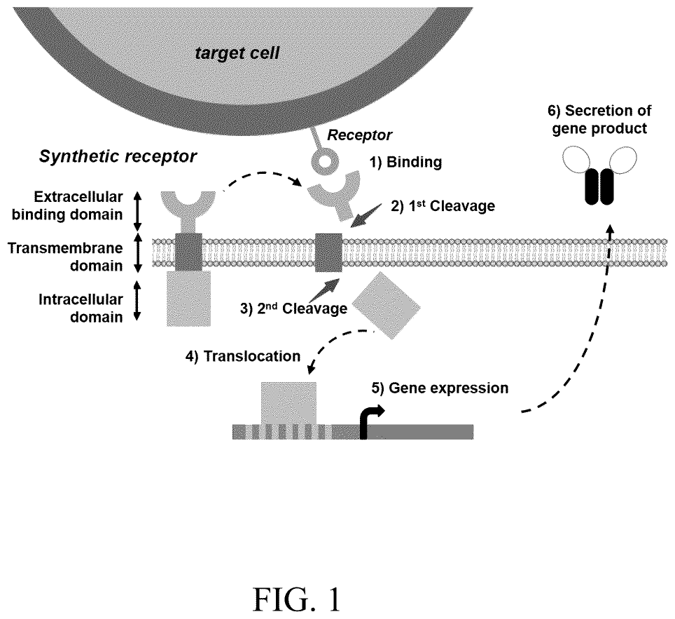

Referring to FIG. 1, a schematic diagram of a synthetic receptor of the present disclosure is provided, with reference to the possibility of a first proteolytic cleavage and a second cleavage after binding of a cognate ligand to the extracellular binding domain. Here, it is shown, in some embodiments, the synthetic receptor extracellular domain binds to a receptor on the target cell. This binding may induce cleavage of the synthetic receptor. The extracellular binding domain may remain bound to the target cell. The intracellular transcription activator domain may be released into the cytoplasm of the cell. The intracellular domain may then translocate into the nucleus, where it may induce expression of a gene, after which the gene product is secreted from the cell.

In some aspects of the disclosure, the extracellular domain may be an antibody or a fragment thereof. In certain embodiments, the extracellular domain also may be a single chain Fv molecule of an antibody that binds glycolipid disialoganglioside (GD2) fused to an Fc region of human IgG1.

The intracellular activity of the method can include at least one fusion protein of a human CTLA4 extracellular domain fused to a wild type or modified Fc region of human IgG. In some embodiments, the IgG may comprise IgG1, IgG2, or IgG4. The intracellular activity also may include at least one transgene encoding a human interleukin. In some embodiments, the human interleukin is human IL2. In certain other embodiments, the intracellular activity includes at least one transgene encoding human IL12. In some embodiments, the at least one transgene may encode any one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40.

Contacting the synthetic receptor with a receptor on the target cell may comprise administering the synthetic receptor to a subject suffering from cancer. Contacting further may include administering the synthetic receptor to a subject suffering from an autoimmune disorder. Contacting further may include administering the synthetic receptor to a subject after an allotransplant or xenotransplant.

### Potential Applications

The synthetic receptors of the present application include many potential applications. By way of illustration and not limitation, the synthetic receptors may serve in allotransplant and xenotransplant patients by recognizing alloantigens and xenoantigens and inducing a tolerogenic response. The receptors of the present disclosure may also act as therapeutics in oncology, recognizing tumor antigens and inducing an immunogenic response (i.e., immune activation). The receptors of the present disclosure also may be useful in regulating autoimmunity by recognizing pro-inflammatory or immune mediators and inducing an anti-inflammatory response (i.e., immune inhibition).

### EXAMPLES

### Example 1: Engineering Proof of Concept

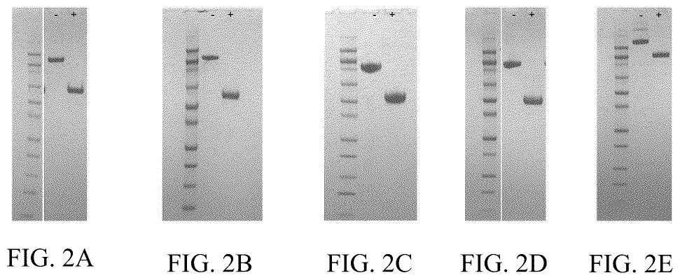

FIG. 2A-2E show results of a polyacrylamide gel electrophoresis demonstrating the production of correctly-sized products from synthetic constructs. FIG. 2A shows a synthetic receptor made of a human CD3-specific single chain Fv molecule fused to the Fc region of human IgG1. FIG. 2B illustrates a synthetic receptor made of a single chain Fv molecule derived from dinutuximab, sold under the trade mark Unituxin®, fused to the Fc region of human IgG1. FIG. 2C shows Belatacept, a fusion protein made of the human CTLA4 extracellular domain fused to the Fc region of human IgG1. FIG. 2D depicts human interleukin 2 fused to the Fc region of human IgG1. FIG. 2E shows an engineered single chain human interleukin 12 molecule fused to the Fc region of human IgG1.

### Example 2: Anti-hCD3 Synthetic Receptor

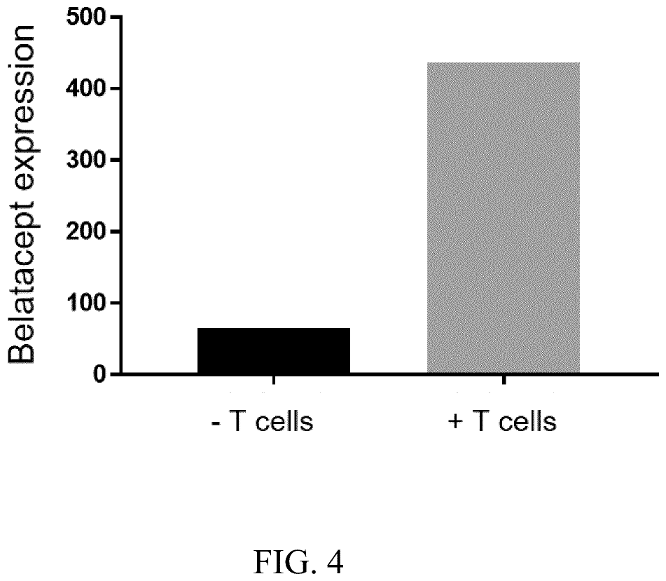

Referring now to FIG. 4, results are shown for an assay for a synthetic receptor with extracellular domain capable of binding human CD3 and responding with the expression and secretion of Belatacept. Here, porcine aortic endothelial cells engineered with the anti-hCD3 synthetic receptor and a responsive transgene encoding Belatacept were exposed to human Jurkat T cells for forty-eight hours. The supernatant from the cells was collected and analyzed for expression of Belatacept. The synthetic receptor only induced expression of Belatacept in the presence of human T cells, which would have the dual benefit of blockade of CD3 and CD80/CD86.

### Example 3: scFV Dinutuximab (Sold Under the Trade Mark Unituxin®) Fused with Fc-IgG1

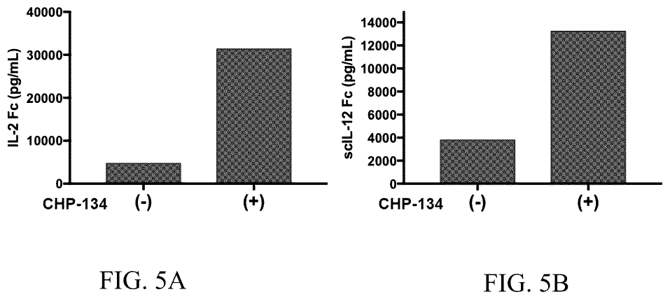

A synthetic receptor was created fusing a single chain Fv derived from dinutuximab with the Fc portion of an IgG1 antibody. This synthetic receptor binds to GD2 on tumor cells and responds with the expression and secretion of human IL-2Fc or human scIL-12Fc. Porcine aortic endothelial cells engineered with the anti-GD2 synthetic receptor and a responsive transgene encoding human IL-2Fc or human scIL-12Fc were exposed to human CHP134 cells for 48 hours. The supernatant from the cells was collected and analyzed for expression of human IL-2Fc or human scIL-12Fc. FIG. 5A and FIG. 5B show the results of the protocol, with IL-2Fc graphically depicted in FIG. 5A and scIL-12Fc graphically depicted in FIG. 5B. The synthetic receptor only induces expression of human IL2-Fc or human scIL12-Fc in the presence of GD2-expressing CHP-134 cells, which would have the dual benefit of blockade of GD2 and production of anti-tumor cytokines.

### Example 4: CTLL-2 Proliferation Assay

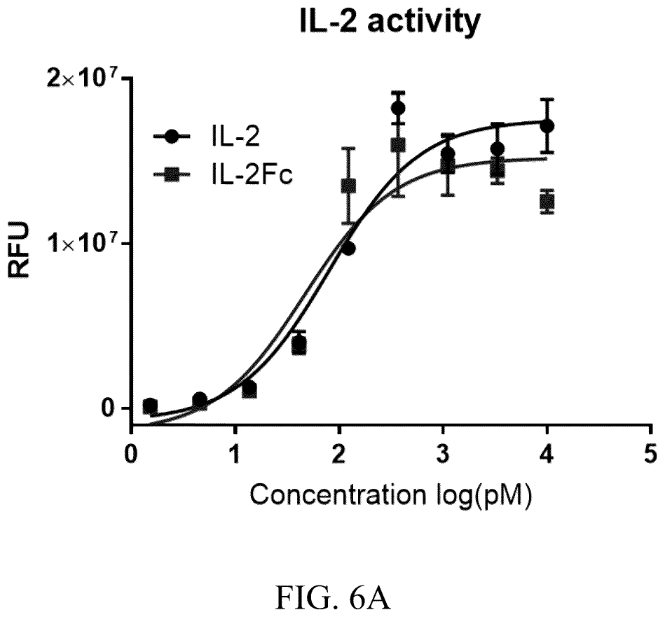

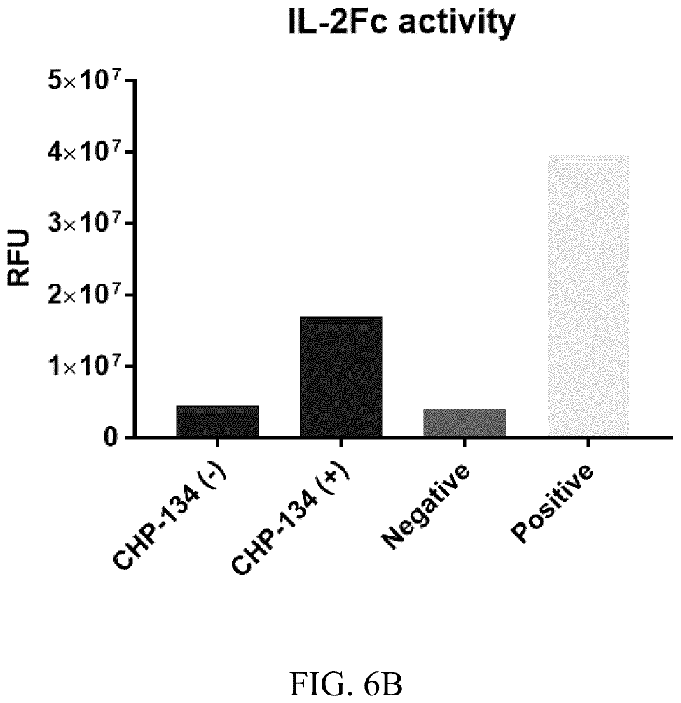

CTLL is a subclone of T cells derived from a C57BL/6 mouse. The cells require IL-2 for growth and are used to assay for its presence in conditioned media and thus may be used to determine the presence of T-cell cytokines by measuring the proliferation of the CTLL-2 cells. In this example, supernatants from porcine aortic endothelial cells engineered with an anti-GD2 synthetic receptor and a responsive transgene encoding human IL-2Fc co-cultured with or without human CHP-134 cells were tested for 48 hours in a CTLL-2 proliferation assay. Results of the assay are shown in FIG. 6A and FIG. 6B. FIG. 6A graphically illustrates that purified engineered human IL2-Fc protein encoded by the transgene functions at levels similar to native human IL2 in a CTLL-2 proliferation assay (RFU refers to relative fluorescence units). FIG. 6B shows IL-2-Fc activity in the CTLL-2 proliferation assay. FIG. 6B graphically illustrates the results of porcine aortic endothelial cells engineered with the anti-GD2 synthetic receptor and a responsive transgene encoding human IL2-Fc cultured for 48 hours with or without GD2-expressing CHP134 cells. In FIG. 6B, CHP-134 (−) refers to culture without CHP-134 cells and CHP-134 (+) refers to culture with CHP-134 cells. “Negative” refers to CTLL-2 cells alone and “Positive” refers to CTLL-2 cells plus purified human IL2-Fc protein. As shown in FIG. 6B, the synthetic receptor only induced expression of IL2-Fc in co-cultures with GD2-expressing human CHP-134 cells, as shown by the ability of the CHP-134 supernatant to stimulate CTLL-2 proliferation.

### Equivalents

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. The attached Appendix is incorporated herein by reference.

We Claim:

1. A method of modulating an activity of a target cell comprising:
contacting a synthetic receptor with a receptor on the target cell, wherein the synthetic receptor comprises at least three domains:
a) an extracellular domain configured to specifically bind to one or more ligands and to optionally release from the synthetic receptor after binding with said ligand,
b) a transmembrane domain derived from a Notch receptor, and
c) an intracellular domain configured to induce expression of a gene when released from the synthetic receptor,
allowing cleavage of the synthetic receptor while the extracellular binding domain remains bound to the target cell and release of the intracellular domain into the nucleus where it induces expression of said gene.

2. The method of claim 1, wherein the wherein the extracellular domain comprises (i) a human CD3-specific single chain Fv molecule fused to the Fc region of human IgG1 or (ii) a single chain Fv molecule derived from dinutuximab fused to the Fc region of human IgG1.

3. The method of claim 1, wherein the extracellular domain comprises an antibody or a fragment thereof.

4. The method of claim 1, wherein the extracellular domain comprises a single chain Fv molecule of an antibody that binds glycolipid disialoganglioside (GD2) fused to an Fc region of human IgG1.

5. The method of claim 1, wherein the intracellular domain comprises at least one fusion protein of a human CTLA4 extracellular domain fused to a wild type or modified Fc region of human immunoglobulin.

6. The method of 5, wherein the human immunoglobulin comprises at least one of IgG1, IgG2, or IgG4.

7. The method of claim 1, wherein the intracellular domain comprises at least one transgene encoding human interleukin.

8. The method of claim 1, wherein the intracellular domain comprises at least one transgene encoding at least one of human interleukin 2 and human interleukin 12.

9. The method of claim 1, wherein contacting comprises administering the synthetic receptor to a subject suffering from cancer.

10. The method of claim 1, wherein contacting comprises administering the synthetic receptor to a subject suffering from an autoimmune disorder.

11. The method of claim 1, wherein contacting comprises administering the synthetic receptor to a subject after the subject has undergone at least one of allotransplant or xenotransplant.
